(19) United States
(12) Patent Application Publication
(10) Pub. No.: US 2009/0232700 A1
(43) Pub. Date: Sep. 17, 2009

(54) Sterilization of Biosensors

(76) Inventors: Javier Alarcon, Durham, NC (US); Kristin Weidemaier, Raleigh, CA (US); David M. Kurisko, Raleigh, NC (US); Srinivasan Sridharan, Bel Air, MD (US)

Correspondence Address: David W. Highet, VP & Chief IP Counsel;Becton, Dickinson and Company, (Morgan, Lewis & Bockius, LLP), 1 Becton Drive, MC 110, Franklin Lakes, NJ, 07417-1880, US

(21) Appl. No.: 12/464,488

(22) Filed: May 12, 2009

Related U.S. Application Data
(62) Division of application No. 11/465,857, filed on Aug. 21, 2006, now abandoned.
(60) Provisional application No. 60/595,942, filed on Aug. 19, 2005.

(51) Int. Cl. A61L 2/08

(52) U.S. Cl. 422/23; 422/22; 422/24; 422/34

(57) ABSTRACT

The present invention relates to methods of making a sterilized biosensor, where the biosensor comprises at least one binding reagent, which comprises at least one non-enzyme proteinaceous binding domain. Certain embodiments of the methods described herein comprise partially assembling the components of the biosensor, except for the binding reagent, and separately sterilizing this partial assemblage and the binding reagent. The sterilized binding reagent and the sterilized partial assemblage are then aseptically assembled to produce the sterilized biosensor. Other embodiments of the methods described herein comprise assembling substantially all of the components of the biosensor, including the binding reagent, and sterilizing the assembled biosensor to produce a sterilized biosensor.

### CROSS-REFERENCE TO RELATED APPLICATIONS

The application is a divisional application of U.S. application Ser. No. 11/465,857, filed 21 Aug. 2006, which claims priority to U.S. Provisional Application No. 60/595,942, filed Aug. 19, 2005, all of which are incorporated by reference herein.

### BACKGROUND OF THE INVENTION

1. Field of the Invention

The present invention relates to methods of making a sterilized biosensor, where the biosensor comprises at least one binding reagent, which comprises at least one non-enzyme proteinaceous binding domain.

2. Background of the Invention

A variety of implantable electrochemical sensors have been developed for detecting and/or quantifying specific agents or compositions in a patient's blood. For instance, glucose sensors are being developed for use in obtaining an indication of blood glucose levels in a diabetic patient. Such readings are useful in monitoring and/or adjusting a treatment regimen which typically includes the regular administration of insulin to the patient. A rapidly advancing area of biosensor development is the use of fluorescently labeled periplasmic binding proteins (PBP's) to detect and quantify analyte concentrations, such as glucose.

All implants must be sterilized before entering the body, and the currently accepted methods of sterilizing implants which comply with AAMI requirements include ionizing radiation, such as gamma radiation, x-ray radiation and electron beam radiation. Additional methods of sterilization include ethylene oxide, ultraviolet light, superheated steam, and filtration.

Because the effects of ionizing radiation depend greatly on protein chemical structure, the dose necessary to produce similar significantly detrimental effects in two different proteins can vary. Radiation effects on the properties of a protein can also be difficult to predict. Radiation normally affects proteins in two competing mechanisms, both resulting from excitation or ionization of atoms. The two mechanisms are chain scission, a random rupturing of bonds, which reduces the molecular weight (i.e., kDa) of the protein, and cross-linking of protein (both intra- and inter-molecular).

The protein's surrounding environment, for example, the presence or absence of oxygen and the post-irradiation storage environment (e.g., temperature and oxygen), may also significantly affect the ratio of scission verses crosslinking during irradiation. Thus, an enzymatic protein such as glucose oxidase may exhibit less post-sterilization effect than a non-enzymatic binding protein such as glucose/galactose binding protein. Although there are published methods of sterilizing proteinaceous biosensors, these biosensors comprise enzymes, such as glucose oxidase, which do not require conformational change for signal transduction. Indeed, the newer, more sophisticated biosensors utilizing PBPs or other proteins that require conformational change for signal transduction may be particularly susceptible to denaturation. Thus, to utilize these newer PBP-based biosensors, methods must be developed for sterilizing the components of the biosensor, while preserving protein function.

### SUMMARY OF THE INVENTION

The present invention relates to methods of making a sterilized biosensor, where the biosensor comprises at least one binding reagent, which comprises at least one non-enzyme proteinaceous binding domain. Certain embodiments of the methods described herein comprise partially assembling the components of the biosensor, except for the binding reagent, and separately sterilizing this partial assemblage and the binding reagent; and then aseptically assembling the sterilized binding reagent with the sterilized partial assemblage to produce the sterilized biosensor. Other embodiments of the methods described herein comprise assembling substantially all of the components of the biosensor, including the binding reagent, and sterilizing the assembled biosensor to produce a sterilized biosensor.

### BRIEF DESCRIPTION OF THE DRAWINGS

FIG. 1 depicts how Qf of a biosensor varies in response to electron-beam sterilization (20 kGy). On the X-axis, lyophilized protein, either without an entrapping matrix (“Solution”) or entrapped in an alginate or PEG matrix, is indicated by a “D.”

FIG. 2 depicts how Qf of a biosensor varies in response to ethylene oxide sterilization. On the X-axis, lyophilized protein, either without an entrapping matrix (“Solution”) or entrapped in an alginate or PEG matrix, is indicated by a “D.”

FIG. 3 depicts how Qf of a biosensor varies in response to gamma sterilization (20 kGy). On the X-axis, lyophilized protein, either without an entrapping matrix (“Solution”) or entrapped in an alginate or PEG matrix, is indicated by a “D.”

FIG. 4 depicts the Qf response of wet and lyophilized pHEMA disks subjected to gamma sterilization for samples with and without the additive trehalose. Samples were prepared with trehalose added at 0, 100, and 500 mg/ml and were exposed to 0 kGy, 10 kGy and 22 kGy of Gamma radiation. The hatched bars on the left represent 5 μm of labeled 3M protein in PBS. The remainder of the X-axis represents either lyophilized or wet pHEMA disks exposed to various doses of radiation, with the labels “0” “100” and “500” representing amounts of trehalose added to the matrix.

### DETAILED DESCRIPTION OF THE INVENTION

The present invention relates to methods of making a sterilized biosensor, where the biosensor comprises at least one binding reagent, which comprises at least one non-enzyme proteinaceous binding domain. The present invention also relates to sterilized biosensor made according to any of the methods described herein. As used herein, “biosensor” is used to mean a composition, device or product that provides information regarding the local biological environment in which the product or composition is located. As used herein, a “biological environment” is used to mean an in vivo, in situ or in vitro setting comprising or capable of supporting tissue, cells, organs, body fluids, single-celled organisms, multicellular organisms, or portions thereof. The cells, tissue, organs or organisms, etc. or portions thereof can be alive (metabolically active) or dead (metabolically inactive). Examples of biological settings include, but are not limited to, in vitro cell culture settings, in vivo settings in or on an organism (such as an implant), a diagnostic or treatment setting, tool or machine, such as a DNA microarray or blood in a dialysis machine. The type of biological environment in which the biosensor can be placed should not limit the present invention.

The biosensors that are sterilized according to the methods of the present invention comprise a binding reagent, with the binding reagent comprising at least one non-enzyme proteinaceous binding domain and at least one signaling moiety. As used herein, a “binding domain” is used herein as it is in the art. Namely, a binding domain is molecule that binds a target in a specific manner. As used herein, a “non-enzyme proteinaceous binding domain” is used to mean an organic compound comprising amino acids that are joined by peptide bonds, but does not detectably catalyze a chemical reaction. Accordingly, the “proteinaceous” aspect of the binding domain may include but is not limited to a bipeptide chain, a tripeptide chain, an oligopeptide chain, a polypeptide chain, a mature protein or protein complex, a lipoprotein, a proteolipid, a glycoprotein, a proteoglycan, and a glycosylphosphatidyl inositol (GPI) anchored protein. Furthermore, the proteinaceous component of the binding domain should not possess the ability to detectably catalyze a chemical reaction. Thus, the binding reagents of the present invention may, for example, comprise non-functional portions of enzymes that may bind a target analyte, but not lower the activation energy required for transforming the analyte into a different chemical entity.

Alternatively, the binding reagents may comprise proteins, or portions thereof, that normally do not catalyze chemical reactions. Examples of such proteins or portions thereof include, but are not limited to, periplasmic binding proteins (PBPs). As used herein a PBP is a protein characterized by its three-dimensional configuration (tertiary structure), rather than its amino acid sequence (primary structure) and is characterized by a lobe-hinge-lobe region. The PBP will normally bind an analyte specifically in a cleft region between the lobes of the PBP. Furthermore, the binding of an analyte in the cleft region will then cause a conformational change to the PBP that makes detection of the analyte possible. Periplasmic binding proteins of the current invention include any protein that possesses the structural characteristics described herein; and analyzing the three-dimensional structure of a protein to determine the characteristic lobe-hinge-lobe structure of the PBPs is well within the capabilities of one of ordinary skill in the art. Examples of PBPs include, but are not limited to, glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein (QBP), oligopeptide binding protein (OppA), or derivatives thereof, as well as other proteins that belong to the families of proteins known as periplasmic binding protein like I (PBP-like I) and periplasmic binding protein like II (PBP-like II). The PBP-like I and PBP-like II proteins have two similar lobe domains comprised of parallel β-sheets and adjacent a helices. The glucose-galactose binding protein (GGBP) belongs to the PBP-like I family of proteins, whereas the maltose binding protein (MBP) belongs to the PBP-like II family of proteins. The ribose binding protein (RBP) is also a member of the PBP family of proteins. Other non-limiting examples of periplasmic binding proteins are listed in Table I.

TABLE I
Genes Encoding Common Periplasmic Binding Proteins

| Gene name | Substrate | Species |
|---|---|---|
| alsB | Allose | E. coli |
| araF | Arabinose | E. coli |
| AraS | Arabinose/fructose/xylose | S. solfataricus |
| argT | Lysine/arginine/ornithine | Salmonella typhimurium |
| artl | Arginine | E. coli |
| artJ | Arginine | E. coli |
| b1310 | Unknown | E. coli |
| | (putative, multiple sugar) | |
| b1487 | Unknown | E. coli |
| | (putative, oligopeptide | |
| | binding) | |
| b1516 | Unknown | E. coli |
| | (sugar binding protein | |
| | homolog) | |
| butE | vitamin B12 | E. coli |
| CAC1474 | Proline/glycine/betaine | Clostridium |
| | | acetobutylicum |
| cbt | Dicarboxylate | E. coli |
| | (Succinate, malate, fumarate) | |
| CbtA | Cellobiose | S. solfataricus |
| chvE | Sugar | A. tumefaciens |
| CysP | Thiosulfate | E. coli |
| dctP | C4-dicarboxylate | Rhodobacter capsulatus |
| dppA | Dipeptide | E. coli |
| FbpA | Iron | Neisseria gonorrhoeae |
| fecB | Fe(III)-dicitrate | E. coli |
| fepB | enterobactin-Fe | E. coli |
| fhuD | Ferrichydroxamate | E. coli |
| FliY | Cystine | E. coli |
| GlcS | glucose/galactose/mannose | S. solfataricus |
| glnH (protein: | Gluconate | E. coli |
| GLNBP) | | |
| gntX | Gluconate | E. coli |
| hemT | Haemin | Y. enterocolitica |
| HisJ (protein: | Histidine | E. coli |
| HBP) | | |
| hitA | Iron | Haemophilus influenzae |
| livJ | Leucine/valine/isoleucine | E. coli |
| livK (protein: | Leucine | E. coli |
| L-BP | | |
| malE (protein: | maltodextrin/maltose | E. coli |
| MBP) | | |
| mglB (protein: | glucose/galactose | E. coli |
| GGBP) | | |
| modA | Molybdate | E. coli |
| MppA | L-alanyl-gamma-D-glutamyl- | E. coli |
| | meso-diaminopimelate | |
| nasF | nitrate/nitrite | Klebsiella oxytoca |
| nikA | Nickel | E. coli |
| opBC | Choline | B. Subtilis |
| OppA | Oligopeptide | Salmonella typhimurium |
| PhnD | Alkylphosphonate | E. coli |
| PhoS (Psts) | Phosphate | E. coli |
| potD | putrescine/spermidine | E. coli |
| potF | Polyamines | E. coli |
| proX | Betaine | E. coli |
| rbsB | Ribose | E. coli |
| SapA | Peptides | S. typhimurium |
| sbp | Sulfate | Salmonella typhimurium |
| TauA | Taurin | E. coli |
| TbpA | Thiamin | E. coli |
| tctC | Tricarboxylate | Salmonella typhimurium |
| TreS | Trehalose | S. solfataricus |
| tTroA | Zinc | Treponema pallidum |
| UgpB | sn-glycerol-3-phosphate | E. coli |
| XylF | Xylose | E. coli |
| YaeC | Unknown | E. coli |
| | (putative) | |
| YbeJ(Gltl) | glutamate/aspartate | E. coli |
| | (putative, superfamily: lysine- | |
| | arginine-ornithine-binding | |
| | protein) | |
| YdcS(b1440) | Unknown | E. coli |
| | (putative, spermidine) | |
| YehZ | Unknown | E. coli |
| | (putative) | |
| YejA | Unknown | E. coli |
| | (putative, homology to | |
| | periplasmic oligopeptide- | |
| | binding protein - Helicobactr | |
| | pylori) | |
| YgiS (b3020) | Oligopeptides | E. coli |
| | (putative) | |
| YhbN | Unknown | E. coli |
| YhdW | Unknown (putative, amino | E. coli |
| | acids) | |
| YliB (b0830) | Unknown (putative, peptides) | E. coli |
| YphF | Unknown (putative sugars) | E. coli |
| Ytrf | Acetoin | B. subtilis |

Other examples of proteins that may comprise the binding domains include, but are not limited to intestinal fatty acid binding proteins (FAPBs). The FABPs are a family of proteins that are expressed at least in the liver, intestine, kidney, lungs, heart, skeletal muscle, adipose tissue, abnormal skin, adipose, endothelial cells, mammary gland, brain, stomach, tongue, placenta, testis, and retina. The family of FABPs is, generally speaking, a family of small intracellular proteins (14 kDa) that bind fatty acids and other hydrophobic ligands, through non-covalent interactions. See Smith, E. R. and Storch, J., J. Biol. Chem., 274 (50):35325-35330 (1999), which is hereby incorporated by reference in its entirety. Members of the FABP family of proteins include, but are not limited to, proteins encoded by the genes FABP1, FABP2, FABP3, FABP4, FABP5, FABP6, FABP7, FABP(9) and MP2. Proteins belonging to the FABP include I-FABP, L-FABP, H-FABP, A-FABP, KLBP, mal-1, E-FABP, PA-FABP, C-FABP, S-FABP, LE-LBP, DA11, LP2, Melanogenic Inhibitor, to name a few.

The invention is not limited by the source organism from the PBPs are isolated. In addition to Table I, which simply illustrates various enzymes isolated from various organisms, other organisms from which PBPs may be isolated include thermophilic and hyperthermophilic organisms. Binding proteins isolated from these thermophilic and hyperthermophilic organisms offer some advantages over binding proteins isolated from mesophilic organisms. In addition to being resistant to high temperatures, proteins isolated from thermophilic and hyperthermophilic have higher resistance to chemical denaturants, are less difficult to purify, and are less susceptible to microbial contamination. Table II provides examples of a few representative organisms wherefrom binding proteins may be isolated.

TABLE II
Examples Thermophilic and Hyperthermophilic
Organisms Harboring PBPs
Thermophilic Organisms

Aeropyrum pernix
Aquifex aeolicus
Bacillus stearothermophilus
Geobacillus kaustophilus
Methanopyrus kandleri
Pyrococcus horikoshii
Pyrococcus abyssi
Sulfolobus solfataricus
Thermoanaerobacter tengcongensis
Thermotoga maritima
Thermotoga neapolitana
Thermococcus kodakaraensis
Thermus thermophilus

The binding domains may be derivative proteins or portions thereof. As used herein, a “derivative” of a protein or polypeptide is a protein or polypeptide that shares substantial sequence identity with the wild-type protein. Examples of derivative proteins include, but are not limited to, mutant and fusion proteins. A “mutant protein” is used herein as it is in the art. In general, a mutant protein can be created by addition, deletion or substitution of the wild-type primary structure of the protein or polypeptide. Mutations include for example, the addition or substitution of cysteine groups, non-naturally occurring amino acids, and replacement of substantially non-reactive amino acids with reactive amino acids. Examples of derivations of PBPs are described in U.S. patent application Ser. No. 10/721,091, filed Nov. 26, 2003, (U.S. Pre-Grant Publication No. 2005/0112685A1), which is hereby incorporated by reference.

As mentioned previously, biosensors must comprise a binding reagent that is able to bind a target analyte in a specific manner. The invention should not be limited by the identity of the analyte; and examples of classes of analytes include, but are not limited to amino acids, peptides, polypeptides, proteins, carbohydrates, lipids, nucleotides, oligonucleotides, polynucleotides, glycoproteins or proteoglycans, lipoproteins, lipopolysaccharides, drugs, drug metabolites, small organic molecules, inorganic molecules and natural or synthetic polymers. As used herein, “carbohydrate” includes, but is not limited to monosaccharides, disaccharides, oligosaccharides and polysaccharides. “Carbohydrate” also includes, but is not limited to, molecules comprising carbon, hydrogen and oxygen that do not fall within the traditional definition of a saccharide—i.e., an aldehyde or ketone derivative of a straight chain polyhydroxyl alcohol, containing at least three carbon atoms. Thus, for example, a carbohydrate may contain fewer than three carbon atoms. As used herein, the term “lipid” is used as it is in the art, i.e., substances of biological origin that are made up primarily or exclusively of nonpolar chemical groups such that they are readily soluble in most organic solvents, but only sparingly soluble in aqueous solvents. Examples of lipids include, but are not limited to, fatty acids, triacylglycerols, glycerophospholipids, sphingolipids, cholesterol, steroids and derivatives thereof. For example, “lipids” include but are not limited to, the ceramides, which are derivatives of sphingolipids and derivatives of ceramides, such as sphingomyelins, cerebrosides and gangliosides. “Lipids” also include, but are not limited to, the common classes of glycerophospholipds (or phospholipids), such as phosphatidic acid, phosphatidylethanolamine, phosphatidylcholine, phosphatidylserine, phosphatidylinositol, phosphatidylglycerol and the like. As used herein, a “drug” can be a known drug or a drug candidate, whose activity or effects on a particular cell type are not yet known. A “drug metabolite” is any of the by-products or the breakdown products of a drug that is changed chemically into another compound or compounds. As used herein, “small organic molecule” includes, but is not limited to, an organic molecule or compound that does not fit precisely into other classifications highlighted herein.

In one embodiment, the biosensor comprises more than one binding domain such that the biosensor can bind to more than one target analyte. In a specific embodiment, all of the target analytes are of the same class of compounds, e.g., proteins, or fatty acids or carbohydrates. In another specific embodiment, at least one of the target analytes is in a different compound class from the other target analytes. For instance, the sterilized biosensor can measure a protein or polypeptide and a carbohydrate or carbohydrates. In yet another specific embodiment of the present invention, none of the target analytes are in the same class of compounds. Furthermore, the target analytes may be specific compounds within a class of compounds, e.g., glucose, palmitate, stearate, oleate, linoleate, linolenate, and arachidonate. Alternatively, the target analytes may be an entire class of compounds, or a portion or subclass thereof, e.g., fatty acids. Specific examples of target analytes include, but are not limited to, glucose, free fatty acids, lactic acid, C-reactive protein and anti-inflammatory mediators, such as cytokines, eicosanoids, or leukotrienes. In one embodiment, the target analytes are fatty acids, C-reactive protein, and leukotrienes. In another embodiment, the target analytes are glucose, lactic acid and fatty acids.

In one aspect of the present invention, the binding reagents to be sterilized according to the methods of the present invention comprise at least one signaling moiety. As used herein a “signaling moiety,” is intended to mean a chemical compound or ion that possesses or comes to possess a detectable non-radioactive signal. Examples of signaling moieties include, but are not limited to, organic dyes, transition metals, lanthanide ions and other chemical compounds. The non-radioactive signals include, but are not limited to, fluorescence, phosphorescence, bioluminescence, electrochemical and chemiluminescence. The spatial relation of the signaling moiety to the binding domain is such that the signaling moiety is capable of indicating a change in the binding domain. Examples of changes in binding domains include, but are not limited to, three-dimensional conformational changes, changes in orientation of the amino acid side chains of non-enzyme proteinaceous binding domains, and redox states of the non-enzyme proteinaceous binding domains. Thus, in one embodiment of the present invention the signaling moiety can, but need not, be attached to the binding domain, for example a GGBP protein, by any conventional means known in the art. For example, the reporter group may be attached via amines or carboxyl residues on the protein. Exemplary embodiments include covalent coupling via thiol groups on cysteine residues of the mutated or native protein.

In one embodiment of the present invention, the binding reagent comprises at least one signaling moiety, where the signaling moiety is a fluorophore. Examples of fluorphores include, but are not limited to fluorescein, coumarins, rhodamines, 5-TMRIA (tetramethylrhodamine-5-iodoacetamide), o-aminobenzoic acid (ABZ), dinitrophenyl (DNP), 4-[(4-dimethylamino)phenyl]-azo)benzoic acid (DANSYL), 5- or 5(6)-carboxyfluorescein (FAM), 5- or 5(6)carboxytetramethylrhodamine (TMR), 5-(2-aminoethylamino)-1-naphthalenesulfonic acid (EDANS), 4-(dimethylamino)azobenzene-4′-carboxylic acid (DABCYL), 4-(dimethylamino)azobenzene-4′-sulfonyl chloride (DABSYL), nitro-Tyrosine (Tyr(NO₂)), Quantum Red™, Texas Red™, Cy3™, 7-nitro-4-benzofurazanyl (NBD), N-((2-iodoacetoxy)ethyl)-N-methyl)am-ino-7-nitrobenzoxadiazole (IANBD), 6-acryloyl-2-dimethylaminonaphthalene (acrylodan), pyrene, Lucifer Yellow, Cy5™, Dapoxyl® (2-bromoacetamidoethyl)sulfonamide, (N-(4,4-difluoro-1,3,5,7-tetramethyl-4-bora-3a,4a-diaza-s-indacene-2-yl)iodoacetamide (Bodipy® 507/545 IA), N-(4,4-difluoro-5,7-diphenyl-4-bora-3a,4a-diaza-s-indacene-3-propionyl)-N′-iodoacetylethylenediamine (BODIPY® 530/550 IA), 5-((((2-iodoacetyl)amino)ethyl)amino)naphthalene-1-sulfonic acid (1,5-IAEDANS), carboxy-X-rhodamine, 5/6-iodoacetamide (XRIA 5,6), eosin, acridine orange, Alexa Fluor 350™, Alexa Fluor 405™, Alexa Fluor 430™, Alexa Fluor 488™, Alexa Fluor 500™, Alexa Fluor 514™, Alexa Fluor 532™, Alexa Fluor 546™, Alexa Fluor 555™, Alexa Fluor 568™, Alexa Fluor 594™, Alexa Fluor 610™, Alexa Fluor 633™, Alexa Fluor 635™, Alexa Fluor 647™, Alexa Fluor 660™, Alexa Fluor 680™, Alexa Fluor 700™ and Alexa Fluor 750™. Other luminescent labeling moieties include lanthanides such as europium (Eu3+) and terbium (Tb3+), as well as metal-ligand complexes of ruthenium [Ru(II)], rhenium [Re(I)], or osmium [Os(II)], typically in complexes with diimine ligands such as phenanthroline. In one particular embodiment of the current invention, there is one labeling moiety per binding domain, and the labeling moieties are acrylodan, NBD and Alexa Fluor 660™. In particular, a FABP is labeled with acrylodan, a GGBP or GGBP derivative specific for glucose is labeled with NBD and a GGBP derivative specific for L-lactate is labeled with Alexa Fluor 660™. Acrylodan-labeled FABP is commercially available (FFA Sciences, LLC, San Diego, Calif.) as “ADIFAB.” A number of binding proteins comprising binding domains that are labeled with fluorescent labeling moieties are disclosed in de Lorimier, R. M. et al., Protein Science 11: 2655-75, (2002), which is herein incorporated by reference.

In another embodiment, the biosensor comprises more than one signaling moiety, where at least one of the additional signaling moieties is a “reference signaling moiety.” The reference signaling moiety should have a luminescence signal that is substantially unchanged upon binding of the target analyte to the binding reagent. “Substantially unchanged” means the luminescence change of the reference signaling moiety is significantly less than the luminescence change undergone by the signaling moiety that indicates ligand binding. The reference signaling moiety, which may comprise luminescent dyes and/or proteins, can be used for internal referencing and calibration. The reference signaling moiety can be attached to any number of components of the device including the binding reagent, the matrix and a component of the biosensor that is not the binding reagent or the matrix, such as, but not limited to, the optical conduit, or a tip.

For the purposes of the present invention, the signal generated by the signaling moiety in response to binding of the binding domain to the analyte must be different than the signal generated by the signaling moiety when analyte is not present. The difference in signals, caused by the presence or absence of analyte binding can be a qualitative difference or a quantitative difference, provided that the differences in the signal are detectable. For example, if the signaling moiety is a fluorophore, the fluorescence intensity may increase or decrease in response to binding of the binding domain to the analyte. A Qf value, defined as the ratio of the luminescent signal at a saturated or infinite ligand concentration (F_{inf}) and the luminescent signal at zero ligand concentration (F0), can be calculated to determine the usefulness of a biosensor utilizing luminescence. Examples of luminescent signals include, but are not limited to, luminescence intensity, a ratio of luminescence intensities, a shift in the luminescence wavelength, an energy transfer efficiency, a luminescence lifetime, or a luminescence polarization. Saturated or infinite ligand concentration may be approximated using a ligand concentration above the equilibrium dissociation constant of the binding domain. A biosensor or binding reagent with a Qf of 1 represents a biosensor/binding reagent with no detectable change in luminescence signal in response to analyte binding. Thus, in one embodiment of the present invention, the methods relate to sterilizing biosensors or binding reagents, where the biosensor or binding reagent retains a Qf of greater than 1. In specific embodiments, the methods of the present invention relate to sterilizing biosensors or binding reagents, where the sterilized biosensor or binding reagent has a Qf of greater than 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 and 10.0 or even greater.

In other embodiments of the invention, the signaling moiety is luminescent, and the luminescence spectrum may undergo a shift in wavelength in response to the analyte. In still other embodiments, the luminescent signal may undergo a change in luminescence lifetime or luminescence polarization in response to the analyte. In one specific embodiment of the present invention, more than one luminescence wavelength is monitored, and the ratio of signal intensities at different wavelengths can change upon binding of the analyte. In the case of ratiometric measurements, a “QR” value is defined as the measured signal ratio at saturating analyte levels, divided by the measured signal ratio in the absence of analyte. Accordingly, the methods of the present invention relate to sterilizing biosensors where the sterilized biosensor has a QR of greater than 1.0. The methods and compositions of the present invention are not limited by the method of measuring analyte binding, or manipulations thereof. Thus, additional methods of quantifying analyte binding using luminescence intensity may be employed without extending beyond the scope of the present invention.

In additional embodiments, the methods of the present invention relate to preserving the luminescent signal responsiveness of a biosensor or a binding reagent, where the methods of preserving luminescence signals comprise entrapping binding reagent within a matrix. As used herein, “preserve” is defined as limiting the loss of luminescence signal responsiveness to at least some degree, such that the Qf value of the sterilized biosensor is greater than 1.0. In specific embodiments, the methods of the present invention relate to preserving at least 5%, 10%, 15%, 20%, 30%, 40%, 50%, 60%, 70%, 80%, 90%, 95% of the luminescence signals of the biosensor after sterilization. Thus other embodiments of the present invention relate to the methods of making a sterilized biosensor, where the binding reagent is entrapped within a matrix.

As used herein, the term “entrap” and variations thereof is used interchangeably with “encapsulate” and is used to mean that the binding reagent is covalently or non-covalently immobilized within or on the constituents of the matrix. The matrix may be comprised of organic material or inorganic material or combinations thereof. Examples of matrices for use in the present methods include but are not limited to, hydrogels and sol-gels. In one embodiment, the matrix may be prepared from biocompatible materials or it may incorporate materials capable of minimizing adverse reactions with the body. The matrix also permits light from optical sources or any other interrogating light to or from the signaling moiety to pass through the biosensor. Adverse reactions for implants include inflammation, protein fouling, tissue necrosis, immune response and leaching of toxic materials. Such materials or treatments are well known and practiced in the art, for example as taught by Quinn, C. P.; Pathak, C. P.; Heller, A.; Hubbell, J. A. Biomaterials 1995, 16(5), 389-396, and Quinn, C. A. P.; Connor, R. E.; Heller, A. Biomaterials 1997, 18(24), 1665-1670.

The matrices may comprise polymers. Suitable polymers which may be used in the present invention include, but are not limited to, one or more of the polymers selected from the group consisting of poly(vinyl alcohol), polyacrylamide, poly(N-vinyl pyrolidone), poly(ethylene oxide) (PEO), hydrolysed polyacrylonitrile, polyacrylic acid, polymethacrylic acid, poly(hydroxyethyl methacrylate), polyurethane polyethylene amine, poly(ethylene glycol) (PEG), cellulose, cellulose acetate, carboxy methyl cellulose, alginic acid, pectinic acid, hyaluronic acid, heparin, heparin sulfate, chitosan, carboxymethyl chitosan, chitin, collagen, pullulan, gellan, xanthan, carboxymethyl dextran, chondroitin sulfate, cationic guar, cationic starch as well as salts and esters thereof. The polymers of the matrix, such as a hydrogel, may also comprise polymers of two or more distinct monomers. Monomers used to create copolymers for use in the matrices include, but are not limited to acrylate, methacrylate, methacrylic acid, alkylacrylates, phenylacrylates, hydroxyalkylacrylates, hydroxyalkylmethacrylates, aminoalkylacrylates, aminoalkylmethacrylates, alkyl quaternary salts of aminoalkylacrylamides, alkyl quaternary salts of aminoalkylmethacrylamides, and combinations thereof. Polymer components of the matrix may, of course, include blends of other polymers. In one particular embodiment of the present invention, the biosensor comprises a matrix, with the matrix comprising a hydrogel of copolymers of (hydroxyethyl methacrylate) and methacrylic acid.

Sol-gel matrices useful for the present invention include material prepared by conventional, well-known sol-gel methods and include inorganic material, organic material or mixed organic/inorganic material. The materials used to produce the sol-gel can include, but are not limited to, aluminates, aluminosilicates and titanates. These materials may be augmented with the organically modified silicates (Ormosils) and functionalized siloxanes, to provide an avenue for imparting and manipulating hydrophilicity and hydrophobicity, ionic charge, covalent attachment of protein, and the like. As used herein the term “hydrolytically condensable siloxane” refers to sol-gel precursors having a total of four substituents, with at least one of the substituents being an alkoxy substituent that is covalently bound to silicone through oxygen and mixtures thereof. In the case of three, two, and one alkoxy substituent precursors, at least one of the remaining substituents may be covalently bound to silicone through carbon.

The matrix may also allow the biosensor to be incorporated at the distal end of a fiber or other small minimally invasive probe to be inserted within the tissue of a patient, to enable an episodic, continuous, or programmed reading to the patient.

The matrix may also comprise one or more additives. For example, one or more additives that may be included in the matrix include, but are not limited to, carbohydrates such as monosaccharides, disaccharides, polysaccharides, amino acids, oligopeptides, polypeptides, proteoglycans, glycoprotein, nucleic acids, oligonucleotides, lipids, fatty acids, natural or synthetic polymers, surfactants, small molecular weight compounds such as antibiotics, drugs or drug candidates, and derivatives thereof. In one particular embodiment, the hydrogel biosensors further comprise at least one carbohydrate or alcohol derivative thereof. More particularly, the matrix may include at least one compound selected from the group consisting of allose, altrose, ascorbate, glucose, mannose, gulose, idose, galactose, talose, ribulose, fructose, sorbose, tagatose, sucrose, lactose, maltose, isomaltose, cellobiose, trehalose, mannitol, sorbitol, xylitol, maltitol, dextrose and lactitol. Without being bound to any theory of mechanism of action, such additives can, for example, provide enhanced storage stability, can prevent or retard degradation, e.g., oxidation, and/or may deter, reduce, or eliminate the detrimental effects of sterilization on the matrix, the binding domain, and/or the label. Additional additives that may be added include surfactants such as those in the TRITON® family or bulking agents, such as, but not limited to, glycine, mannitol, lactose monohydrate, and povidone K-12. Other additives that may be added to the matrix, binding domain, and/or label include, but are not limited to hindered amine (or amide) stabilizers or other free radical scavengers, antioxidants, benzophenones, and benzotriazoles. In one embodiment the hindered amine/amide stabilizers, such as the 2,2,6,6-tetraalkyl-4-piperidyl class of compounds are used. For example, commercially available piperidyl additives Ciba® CHIMASSORB® 944: poly[[6-[(1,1,3,3-tetramethylbutyl)amino]-1,3,5-triazine-2,4-cliyl][(2,2,6,6-tetramethyl-4-piperidinyl)imino]-1,6-hexanediyl[(2,2,6,6-tetramethyl-4-piperidinyl)imino]]) CAS No. [71878-19-8]; Ciba® TINUVIN® 770: bis(2,2,6,6-tetramethyl-4-piperidyl)dodecanoate [piperidyl sebacate]; Ciba® TINUVIN® 622: butanedioic acid, dimethylester, polymer with 4-hydroxy-2,2,6,6-tetramethyl-1-piperidine ethanol, CAS No. [65447-77-0]; and Great Lakes Chemical Uvasil 299: polymethyl propyl-3-oxy[4(2,2,6,6-tetramethyl)piperidinyl]siloxane may be used. Examples of antioxidants or free radical scavengers that may also be useful include quinones, e.g., 1,4-benzenediol, and hydroquinone mono ethylether aromatic ketones, e.g., 1,3-Diphenyl-2-propanone, vitamins and metals. Specific examples of antioxidants include but are not limited to vitamin E, beta-carotene, vitamin C, selenium, human thiol-specific antioxidant protein 1 (hTSAP1), methionine, heme-oxygenase-1 (HO-1) and ferritin to name a few. In addition, particular compounds, such as calcium, can be added to the matrix, with or without the protein, or to the protein itself to stabilize the binding domain or matrix. Any combination of the above mentioned additives are also envisaged. Additionally, the additives may be added to the matrix with or without the binding domain or to the binding domain in either a dry or wet form. The order of addition of the additives or the portion of the biosensor to which it is added is not to be construed as limiting.

As mentioned above, the binding molecule may be entrapped within a matrix, such as a hydrogel, which may then be used as an implantable device. The biosensor comprising binding domain can be in any desirable form or shape including one or more of disk, cylinder, patch, nanoparticle, microsphere, porous polymer, open cell foam, and combinations thereof, providing the biosensor is permeable to the analyte.

In one embodiment, the methods of the present invention relate to making a sterilized biosensor, with the methods comprising assembling at least a portion of the biosensor, where the assembled portion does not include the binding reagent, and sterilizing this partial assemblage. Separately, the binding reagent is sterilized; and the sterilized binding reagent and partial assemblage are aseptically assembled to produce the sterilized biosensor. In a specific embodiment, the process of assembling the sterilized binding reagent and the sterilized partial assembly to each other comprises entrapping the binding reagent in a matrix, where the matrix is part of the partial assemblage. Methods of entrapping the binding reagent within a matrix are described in U.S. patent application Ser. No. 11/077,028, filed Mar. 11, 2005, and published as United States Pre-grant Publication 2005/0239155, which is hereby incorporated by reference.

The methods of sterilizing the assembled biosensor, partially assembled biosensor or the individual components thereof, should not limit the scope of the invention. Examples of methods of sterilizing the biosensor include, but are not limited to, dialysis, irradiation, ultraviolet light, filtration, chemical treatment (e.g., using ethylene oxide “ETO” or hydrogen peroxide), or other known sterilization methods, such as, but not limited to, superheated steam sterilization (autoclaving). Methods of sterilization via irradiation are well-known in the art, and include electron beam sterilization, x-ray sterilization, ultraviolet light, beta radiation and gamma (e.g., ⁶⁰Co and ¹³⁷Cs) radiation. In one embodiment, electron beam sterilization is performed with a single dose of 2.0 Mrads or greater (or 20 kGy or greater). In other embodiments, smaller dose levels may be used if sufficient sterilization may be achieved at the lower dose, such as for example 1-2 Mrads (10-20 kGy). The level of sterilization of the biosensor can be measured using standard techniques governed by ANSI/AAMI/ISO 11137-1995 “Sterilization of health care products—Requirements for validation and routine control—Radiation sterilization,” which is incorporated by reference. In one embodiment of the present invention, the biosensor has a sterility-assurance level (SAL) of at least 1×10⁻³. Sterility assurance level (SAL) is used herein as it is in the art, namely it is defined as the probability of an item being nonsterile after going through a validated sterilization process. For example, an SAL of 1×10⁻³ means that the probability of an item being non-sterile is 1 in 1000, after sterilization using a validated sterilization process. In additional embodiments, the biosensor has an SAL of at least 1×10⁻⁴, 1×10⁻⁵ or 1×10⁻⁶ (e.g., probability of being non-sterile is 1 in one million). Other, more specific doses of radiation can be determined, based upon the components of the biosensor and include, but are not limited to such doses as 1 kGy or less, 2 kGy, 3 kGy, 4 kGy, 5 kGy, 6 kGy, 7 kGy, 8 kGy, 9 kGy, 10 kGy, 12 kGy, 15 kGy, 20 kGy, 25 kGy, 30 kGy, 35 kGy, 40 kGy, 45 kGy and 50 kGy or even more. In certain specific embodiments, the biosensor is sterilized in accordance with ANSI/AAMI/ISO 11137-1995 “Sterilization of health care products—Requirements for validation and routine control—Radiation sterilization” and also ISO 13408 “Aseptic processing of healthcare products” which is hereby incorporated by reference.

In another embodiment, the sterilization process comprises irradiation in an environment designed to minimize oxidation of the sensor components. For example, the sensor can be sterilized in an inert gas environment to maintain low oxygen levels. In a specific example, the binding reagent is irradiated in the presence of at least one inert gas. Gases designed to minimize, reduce, or prevent oxidation of sensor components include, but are not limited to Helium (He), Neon (Ne), Argon (Ar), Krypton (Kr), Xenon (Xe), and Nitrogen (N₂). Other methods for maintaining a low oxygen environment during sterilization include vacuum packaging or packaging in the presence of oxygen scavengers such as powdered iron oxide.

The binding reagent, comprising a non-enzyme proteinaceous binding domain, may be sterilized separately from the remaining components of the biosensor. Methods of sterilizing proteinaceous compounds include but are not limited to filter sterilization and additional methods of sterilization described herein.

In another embodiment, the methods of the present invention relate to making a sterilized biosensor, with the biosensor comprising at least one binding reagent that is itself comprised of at least one non-enzyme proteinaceous binding domain. These particular methods comprise assembling at least some of the components of the biosensor, including the binding reagent, and sterilizing the biosensor. In specific embodiments, the process of assembling the biosensor, including the binding reagent, comprises entrapping the binding reagent within a matrix.

In another embodiment of the present invention, the methods of the present invention comprise a drying process. Examples of drying processes include any process designed to remove water, such as, but not limited to, lyophilization, heat, vacuum, inert gas, dessication, dry air, spray drying, combinations thereof, or any process designed to remove water or volatile solvents. In one embodiment, the drying process is lyophilization. In one specific embodiment, the biosensor, including the binding domain, is assembled and lyophilized prior to sterilization. In another embodiment, the binding domain is lyophilized prior to assembly into the biosensor. In essence, this particular aspect of the invention should not be limited by the point in time when the binding domain is dried. Methods of drying, including lyophilization, are well-known in the art. The assembled biosensor that is dried may or may not comprise a matrix with additives. In yet another embodiment of the present invention, the biosensor, including the binding domain, is assembled and vacuum dried prior to sterilization. Methods of vacuum drying are well known in the art. The assembled biosensor that is vacuum dried may or may not comprise a matrix with additives. Additional methods of drying include but are not limited to spray freeze drying and inert gas drying.

In additional embodiments, the methods of the present invention relate to preserving the luminescence signal responsiveness of a biosensor or a binding reagent, where the methods of preserving luminescence signal comprise entrapping binding reagent within a matrix and lyophilizing the matrix (entrapping a binding reagent), prior to sterilization. Thus other embodiments of the present invention relate to the methods of making a sterilized biosensor, where the binding reagent is entrapped within a matrix and subsequently lyophilized.

In another embodiment of the present invention, the biosensor is assembled and packaged. The packaging materials should be resistant to microbial migration and include, but are not limited to, tyvek, tyvek/mylar foil, foil, foil laminate and poly/mylar/polyethylene laminate pouches. The packaging material may be configured as “blister pack” or form/fill/seal packages.

The present invention also relates to sterilized binding reagents, where the binding reagent comprises at least one non-enzyme proteinaceous binding domain entrapped in a matrix, where the binding domain is capable of changing its three-dimensional conformation upon specific binding to an analyte.

The examples herein are provided to illustrate select embodiments of the present invention and are not intended to limit the scope of the invention.

### EXAMPLES

### Example 1

### Preparation of Alginate Disks and PEG Disks Containing a Binding Protein Entrapped in a Matrix

A fluorescent-labeled triple mutant of GGBP (“the 3M protein”) was prepared as follows. The 3M protein is a GGBP protein (GenBank Accession No. P02927, without the 23 amino acid leader sequence), and where a cysteine is substituted for an glutamic acid at position 149, an arginine is substituted for an alanine at position 213, and a serine is substituted for leucine at position 238 (E149CA213RL238S). The 3M protein was labeled with IANBD, and the NBD-labeled 3M protein was prepared as described in U.S. application Ser. No. 10/040,077, filed Jan. 4, 2002, now U.S. Pat. No. 6,855,556, and Ser. No. 11/077,028, filed Mar. 11, 2005, and published as United States Pre-grant Publication 2005/0239155 both of which are incorporated herein by reference.

Alginate disk were prepared in the following manner. A mix of 2% Alginate in sterile water by weight was prepared. To this solution we added 0.1 M of 1-hydroxy benzo triazole (HOBT) and 0.1M of Adipic acid dihydraze (ADD). Both solutions were prepared in MES buffer and pH was adjusted to 6.5. After homogenization, 9.8 mg of 1-ethyl-3-(3-dimethylamino-propyl) carbodiimide (EDC) in 50 uL of 100 mM MES and 0.5 mL of 400 mM N-hydroxysuccinimide (NHS) were added to the Alginate solution. After mixing, the solution was poured in between two glass plates separated by an approximately 1 mm spacer. After at least about two hours, the alginate sheet was removed from between the plates, and was cut into circular disks using a biopsy punch. The disks can be stored in PBS until further use.

After the Alginate disks were cut, they were put in a solution of 1M Ethanolamine for about 15 minutes, and subsequently washed in phosphate buffer solution (PBS) for about 30 minutes. A 50 μM solution of the 3M protein in PBS was then leached into the alginate disks overnight by placing the disks in the protein solution. After overnight leaching, the disks were rinsed with PBS and then placed in a solution of 100 mM EDC in MES and 400 mM NHS for about 40 minutes. The disks were subsequently placed in a 1M solution of ethanolamine in water for about 30 minutes, after which they were washed and stored in PBS.

Poly(ethylene glycol) (PEG) hydrogel disks were created in the following manner. 400 mg of 8-arm amino terminated PEG was mixed with 200 mg of poly ethylene glycol-Bis-Benzotriazolyl Carbonate (Bi BTC) in 1.8 mL of NHS in water. A 50 μM solution of the 3M protein was added to this solution. When all the components were together, the final mix was placed between two glass plates separated by an approximately 1 mm and allowed to set. After at least about one hour, the PEG/3M hydrogel sheet was removed from between the plates, and was cut into circular disks using a biopsy punch. The disks can be stored in PBS until further use.

Some of the disks were lyophilized by placing them in a −70° C. freezer and subsequently dried in a lyophilizer. The non-lyophilized disks are herein referred to as “wet” disks, whereas the lyophilized disks are herein referred to as “dried” disks.

### Example 2

### Electron-Beam Sterilization of Non-Lyophilized and Lyophilized Disks as Prepared in Example 1

The wet and dry disks of Example 1 were sterilized using electron-beam radiation. In addition, protein in solution and lyophilized protein were also irradiated using electron-beam radiation. In this experiment, the 20 kiloGrays (2 Mrads) (6.25 kGy/sec) were used, and the dose was confirmed by dosimeter.

### Example 3

### Gamma Radiation Sterilization of Non-Lyophilized and Lyophilized Disks as Prepared in Example 1

The wet and dry disks of Example 1 were sterilized using gamma radiation. In addition, lyophilized and non-lyophilized protein in solution was also irradiated using gamma radiation. In this experiment, the 20 kiloGrays (2 Mrads) was used. In this experiment, the 20 kiloGrays (2 Mrads) (8.33 kGy/hr) were used, and the dose was confirmed by dosimeter.

### Example 4

### Ethylene Oxide Sterilization of Non-Lyophilized and Lyophilized Disks as Prepared in Example 1

The wet and dry disks of Example 1 were sterilized using ethylene oxide (ETO). In addition, protein in solution and lyophilized protein were also irradiated using ethylene oxide. In this experiment, the disks or protein were exposed to ETO for 2 hours at about 6° C.

### Example 5

### Responsiveness of Biosensor Disks after Sterilization

The glucose responsiveness of the sterilized disks was tested. The biosensors were placed in the wells of a black 96 well plate along with 180 μL PBS buffer per disk, and the initial fluorescence intensities (F₀) were measured using a CytoFluor fluorescence multi-well plate reader (excitation and emission filters were centered at 485 nm and 530 nm, respectively). Next, 20 μL of 1 M glucose/water solution was added into each well, providing a final glucose concentration of 100 mM. The fluorescence intensity changes were recorded again after the solution was equilibrated for 20 minutes to allow glucose to completely diffuse into the sterilized disks and bind with the binding reagent. Here, and in the following examples, the protein binding response is defined as a change in fluorescence intensity, Qf, which is the ratio of the fluorescence intensity of the biosensor disks in the presence of 100 mM (near saturating) glucose concentration to the fluorescence intensity of the hydrogel biosensor disks in the absence of glucose.

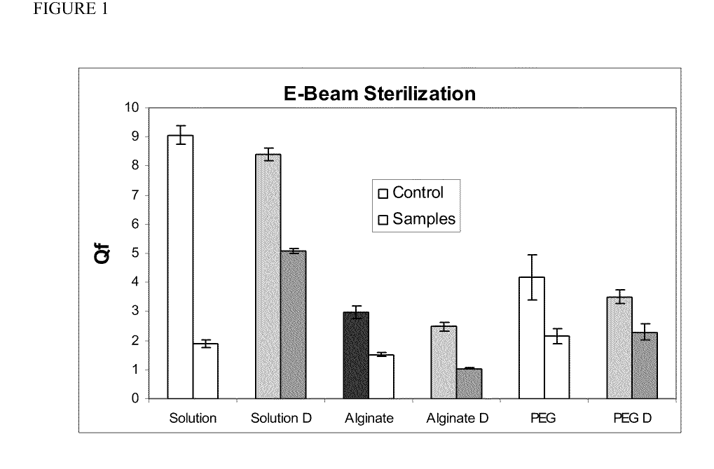

FIG. 1 shows how Qf varies in response to electron-beam sterilization (20 kGy). Specifically, The unsterilized NBD-labeled 3M protein in free solution had a Qf of approximately 9.1, whereas the sterilized NBD-labeled 3M protein in free solution had a Qf of approximately 1.9. The unsterilized lyophilized NBD-labeled 3M proteins in free solution had a Qf of approximately 8.4, whereas the lyophilized sterilized NBD-labeled 3M protein in free solution had a Qf of approximately 5.1.

The unsterilized NBD-labeled 3M protein entrapped in alginate had a Qf of approximately 3.0, whereas the sterilized NBD-labeled 3M protein entrapped in alginate had a Qf of approximately 1.5. The unsterilized lyophilized NBD-labeled 3M proteins entrapped in alginate had a Qf of approximately 2.5, whereas the lyophilized sterilized NBD-labeled 3M protein entrapped in alginate had a Qf of approximately 1.0.

The unsterilized NBD-labeled 3M protein entrapped in PEG had a Qf of approximately 4.2, whereas the sterilized NBD-labeled 3M protein entrapped in PEG had a Qf of approximately 2.2. The unsterilized lyophilized NBD-labeled 3M proteins entrapped in PEG had a Qf of approximately 3.5, whereas the lyophilized sterilized NBD-labeled 3M protein entrapped in PEG had a Qf of approximately 2.3.

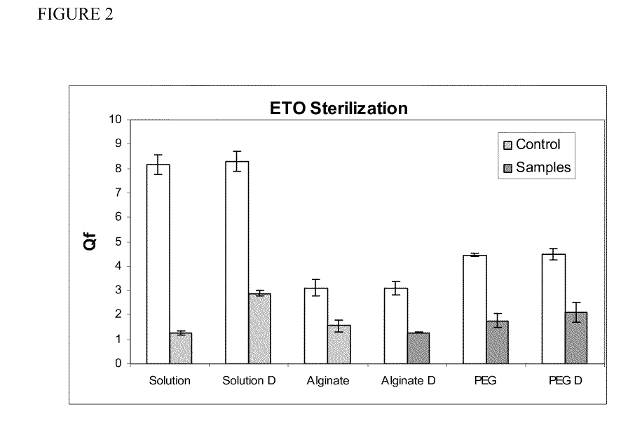

FIG. 2 shows how Qf varies in response to ethylene oxide (ETO) sterilization. Specifically, The unsterilized NBD-labeled 3M protein in free solution had a Qf of approximately 8.2, whereas the sterilized NBD-labeled 3M protein in free solution had a Qf of approximately 1.3. The unsterilized lyophilized NBD-labeled 3M proteins in free solution had a Qf of approximately 8.3, whereas the lyophilized sterilized NBD-labeled 3M protein in free solution had a Qf of approximately 2.9.

The unsterilized NBD-labeled 3M protein entrapped in alginate had a Qf of approximately 3.1, whereas the sterilized NBD-labeled 3M protein entrapped in alginate had a Qf of approximately 1.6. The unsterilized lyophilized NBD-labeled 3M proteins entrapped in alginate had a Qf of approximately 3.1, whereas the lyophilized sterilized NBD-labeled 3M protein entrapped in alginate had a Qf of approximately 1.3.

The unsterilized NBD-labeled 3M protein entrapped in PEG had a Qf of approximately 4.5, whereas the sterilized NBD-labeled 3M protein entrapped in PEG had a Qf of approximately 1.8. The unsterilized lyophilized NBD-labeled 3M proteins entrapped in PEG had a Qf of approximately 4.5, whereas the lyophilized sterilized NBD-labeled 3M protein entrapped in PEG had a Qf of approximately 2.1.

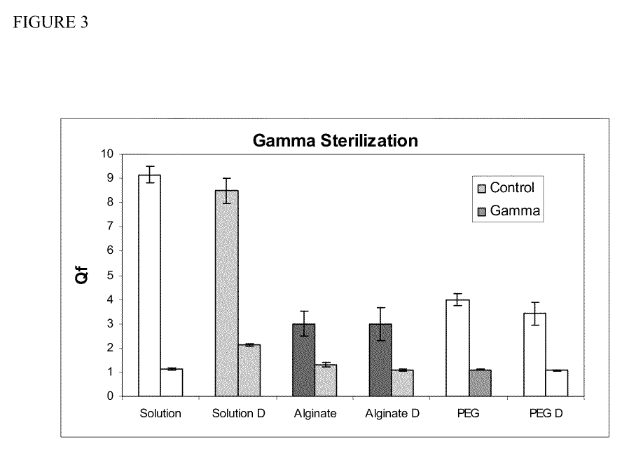

FIG. 3 shows how Qf varies in response to gamma radiation sterilization (20 kGy). Specifically, The unsterilized NBD-labeled 3M protein in free solution had a Qf of approximately 9.2, whereas the sterilized NBD-labeled 3M protein in free solution had a Qf of approximately 1.2. The unsterilized lyophilized NBD-labeled 3M proteins in free solution had a Qf of approximately 8.5, whereas the lyophilized sterilized NBD-labeled 3M protein in free solution had a Qf of approximately 2.1.

The unsterilized NBD-labeled 3M protein entrapped in alginate had a Qf of approximately 3.0, whereas the sterilized NBD-labeled 3M protein entrapped in alginate had a Qf of approximately 1.3. The unsterilized lyophilized NBD-labeled 3M proteins entrapped in alginate had a Qf of approximately 3.0, whereas the lyophilized sterilized NBD-labeled 3M protein entrapped in alginate had a Qf of approximately 1.1.

The unsterilized NBD-labeled 3M protein entrapped in PEG had a Qf of approximately 4.0, whereas the sterilized NBD-labeled 3M protein entrapped in PEG had a Qf of approximately 1.1. The unsterilized lyophilized NBD-labeled 3M proteins entrapped in PEG had a Qf of approximately 3.4, whereas the lyophilized sterilized NBD-labeled 3M protein entrapped in PEG had a Qf of approximately 1.1.

### Example 6

### Qf of Biosensor Disks in Response to Varying Levels of Gamma Irradiation

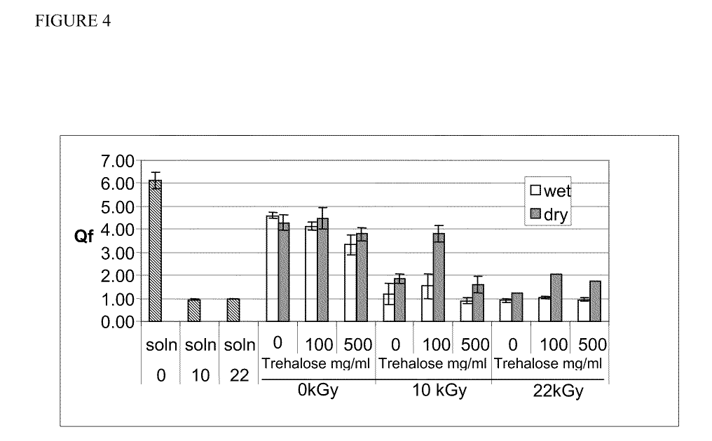

Disks of poly(hydroxyethyl methacrylate) (poly HEMA) with varying concentrations of Trehalose (100 mg/ml or 500 mg/ml) were prepared with covalently-immobilized (c.i.) NBD-3M protein. Poly HEMA disk preparation consisted of 20% HEMA monomer, 9 moles HEMA: 1 mole MAA, 2% PEGDMA, in DMF, with overnight polymerization at 70° C. The disks were punched from the slab with a 4-mm biopsy punch and subsequently, disks were infused with 12 uM NBD-3M in 0.1 M MES (pH 6.5) which was covalently immobilized with 2.5 mM EDC and 0.62 mM NHS for 4 hr. This solution was then replaced with 1M ethanolamine (pH 8.5) for 1 hr to stop further crosslinking. The disks were then washed 2× in PBS, disks were then placed in 30 ml of 0, 100, or 500 mg trehalose/ml of PBS at 4° C. for 3 days. After three days half the disks were lyophilized and half were kept in PBS at 4° C. In addition, control disks (poly HEMA with immobilized NBD-3M without Trehalose) were also prepared. Disks (wet, lyophilized, and control) were placed in microfuge tubes (2 disks/tube) and subjected to gamma (Cobalt 60) irradiation along with 5 μM NBD-3M solution. Gamma irradiation was at 10 kGy (6.66 kGy/hour) and 22 kGy (11 kGy/hour). (10 disks at each trehalose concentration/storage condition). After radiation the disks were challenged with 0 mM or 100 mM glucose and fluorescence measured at each concentration to obtain the protein activity as measured by Qf (F100 mM/F0 mM). In this experiment, the dose was confirmed by dosimeter. As seen in FIG. 4, the addition of trehalose led to increased protein activity as exhibited by Qf values of greater than 1 at radiation doses of 10 and 22 kGy, particularly in the lyophilized samples.

### Example 7

### Aseptic Assembly of Biosensor

One embodiment of the methods of the present invention provides methods to produce a sterile sensor by aseptically assembling subassemblies that have been previously sterilized, e.g. by irradiation. Briefly, an alginate hydrogel matrix was applied to a sensor device comprising a 400 micron core-diameter glass fiber housed in a 21 gauge steel needle. The glass surface of the fiber was amine functionalized with 3′-aminopropyltrimethoxy silane via a plasma treatment process. An alginate hydrogel matrix was then applied and covalently cross-linked through the carboxyls with adipic acid dihydrazide (AAD), via carbodiimide chemistry. One example of the device that was sterilized is described in U.S. patent application Ser. No. 10/967,221, filed Oct. 19, 2004 (United States Pre-Grant Publication No. 2005/0113658), the entirety of which is incorporated by reference. The device was then packaged and subjected to terminal sterilization by e-beam radiation at a dose of about 23 kGy. The dose was verified by dosimeter. After e-beam sterilization, the sensors with matrix were then transferred into a class 100 clean room. A fluorescent-labeled triple mutant of GGBP (“the 3M protein”), as described in Example 1, was infused into the device and covalently attached to the matrix using aseptic handling techniques. The sensor was then repackaged into packaging components that had been previously sterilized by e-beam irradiation. Sterility of the final devices was confirmed by validation of the process via bioburden estimations and dose verifications, per AAMI/ISO Standard 11137 “Sterilization of Healthcare Products—Requirements for validation and routine control-Radiation Sterilization,” as well as through sterility testing of three consecutive lots to validate the aseptic process per ISO 13408 “Aseptic processing of healthcare products.”

Table III shows the Qf values of the sterilized sensors compared to control sensors that had not undergone e-beam sterilization of the matrix. The values in each group represent the averages of 20 sensors. As can be seen from the data, the sterilized sensors have similar protein activity compared to control (unsterilized) sensors.

TABLE III
3M-NBD/Alignate Sensors

| | | Sensors |
|---|---|---|
| | Sensors | Matrix Ebeam |
| | Matrix No Sterilization | Sterilized(20 kGy) |
| Average Qf | 6.34 | 5.34 |
| Standard Deviation | 0.43 | 0.96 |

What is claimed is:

1. A method of making a sterilized biosensor, said method comprising
a) assembling components of said biosensor, said biosensor comprising at least one binding reagent, said binding reagent comprising at least one non-enzyme proteinaceous binding domain entrapped in a matrix, to produce an unsterilized biosensor; and
b) sterilizing said assembled biosensor, said sterilization of said assembled biosensor comprising a type of sterilization selected from the group consisting of electron beam radiation, gamma radiation and ethylene oxide; and,
wherein said assembled, sterilized biosensor is capable of providing accurate concentration measurements of at least one analyte.

2. The method of claim 1, wherein at least one analyte is glucose.

3. The method of claim 1, wherein said sterilization type of said assembled biosensor comprises a type of sterilization selected from the group consisting of filtration, electron beam radiation, gamma radiation, ethylene oxide, ultraviolet and hydrogen peroxide.

4. The method of claim 3, wherein said sterilization type is electron beam radiation and comprises a dose of at least 5 kGy.

5. The method of claim 4, wherein said electron beam radiation is performed in the presence of at least one inert gas.

6. The method of claim 1, wherein said non-enzyme proteinaceous binding domain is selected from the group consisting of periplasmic binding proteins, fatty acid binding proteins and derivatives thereof.

7. The method of claim 6 wherein said non-enzyme proteinaceous binding domain is a periplasmic binding protein.

8. The method of claim 7, wherein said periplasmic binding protein is selected from the group consisting of glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein (QBP), oligopeptide binding protein (OppA) and derivatives thereof.

9. The method of claim 8, wherein said non-enzyme proteinaceous binding domain is a derivative of GGBP.

10. The method of claim 6, wherein said non-enzyme proteinaceous binding domain is entrapped in a matrix, said matrix selected from the group consisting of a hydrogel and a sol-gel.

11. The method of claim 10, wherein said matrix is a hydrogel and wherein said hydrogel matrix comprises one or more of the polymers selected from the group consisting of poly(vinyl alcohol), polyacrylamide, poly(N-vinyl pyrolidone), poly(ethylene oxide) (PEO), hydrolysed polyacrylonitrile, polyacrylic acid, polymethacrylic acid, poly(hydroxyethyl methacrylate), polyurethane polyethylene amine, poly(ethylene glycol) (PEG), cellulose, cellulose acetate, carboxy methyl cellulose, alginic acid, pectinic acid, hyaluronic acid, heparin, heparin sulfate, chitosan, carboxymethyl chitosan, chitin, collagen, pullulan, gellan, xanthan, carboxymethyl dextran, chondroitin sulfate, cationic guar, cationic starch and salts and esters thereof.

12. The method of claim 11, wherein said hydrogel matrix further comprises an additive selected from the group consisting of allose, altrose, ascorbate, glucose, mannose, gulose, idose, galactose, talose, ribulose, fructose, sorbose, tagatose, sucrose, lactose, maltose, isomaltose, cellobiose, trehalose, mannitol, sorbitol, xylitol, maltitol, dextrose and lactitol.

13. The method of claim 10, wherein said matrix is dried.

14. The sterilized biosensor of claim 1, wherein said sterilized biosensor has a sterility assurance level (SAL) of at least 1×10⁻³.

15. The sterilized biosensor of claim 14, wherein said sterilized biosensor has a sterility assurance level (SAL) of at least 1×10⁻⁶.

16. A method of increasing or preserving the luminescence signal responsiveness of a sterilized biosensor, said biosensor comprising at least one binding reagent, said binding reagent comprising at least one non-enzyme proteinaceous binding domain, said method comprising at least one step selected from the group consisting of: (a) entrapping said binding reagent in a matrix prior to sterilizing said binding reagent, and (b) drying said binding reagent prior to sterilizing said binding reagent.

17. A sterilized binding reagent comprising at least one sterilized non-enzyme proteinaceous binding domain entrapped in a matrix, said sterilized binding domain being capable of changing three-dimensional conformations upon binding an analyte.
